(19) United States
(12) Patent Application Publication
(10) Pub. No.: US 2023/0063247 A1
(43) Pub. Date: Mar. 2, 2023

(54) PERSONALIZED RECOMMENDATION SYSTEM

(71) Applicant: Siemens Medical Solutions USA, Inc., Malvern, PA (US)

(72) Inventors: Sailesh Conjeti, Erlangen (DE); Philipp Hoelzer, Plainsboro, NJ (US); Ingo Schmuecking, Yardley, PA (US); Anna Jerebko, Paoli, PA (US); Luca Bogoni, Philadelphia, PA (US); Gerardo Hermosillo Valadez, West Chester, PA (US); Valentin Ziebandt, Nürnberg (DE)

(21) Appl. No.: 17/411,159

(22) Filed: Aug. 25, 2021

(51) Int. Cl. G16H 50/20; G16H 40/20; G16H 30/20; G16H 30/40; G16H 10/60; G06N 3/04; G06N 3/08

(52) U.S. Cl. CPC G16H 50/20; G16H 40/20; G16H 30/20; G16H 30/40; G16H 10/60; G06N 3/04; G06N 3/08

(57) ABSTRACT

A framework for personalized recommendation. An image content profile for a current case is generated. One or more auxiliary information representations associated with the current case are further generated. Affinity scores for radiology service providers are then determined by applying service profiles of the radiology service providers, the image content profile and the one or more auxiliary information representations to a trained recommendation engine. The current case is then assigned to one of the radiology service providers based on the affinity scores.

### TECHNICAL FIELD

The present disclosure generally relates to medical data processing, and more particularly to a personalized recommendation system.

### BACKGROUND

A major part of radiology workflow is image interpretation and reporting of imaging procedures. Large radiology practices may perform a high volume of imaging procedures per year and have one hundred or more radiologists engaged in reporting. In an efficient workflow, radiologists can complete a case and immediately proceed to reading the next case. However, given the broad scope of radiology imaging and range of clinical situations, there are numerous and often competing requirements to be considered when deciding which case to read next and who is the appropriate and available radiologist to read the case. When radiologists spend substantial time on determining which case to read or start image interpretation and then interrupt with a different case, the workflow efficiency is reduced and the risk of radiologist burnout increases.

In large hospital systems and multi-specialty radiology practices, worklists are used to manage the reading workflow of the radiologist. The worklist contains studies or cases assigned to a particular radiologist for reading. The worklist traditionally follows a “First-In First-Out” queuing methodology, with studies prioritized on a first-in, first-out basis without any use of smart case assignment.

Workflow orchestration was introduced as a method of rule-based assignment of cases to the most appropriate radiologist available and efficiently balance caseloads to maximize quality and productivity, while fulfilling service level agreements with referring physicians for report turnaround time. Radiologists are assigned cases in a worklist based on their training and experience (e.g., general, sub-specialty), defined workload, origin of images, prioritization, load balancing, etc. However, such solutions do not provide smart assignment of corresponding cases to appropriate radiologists.

### SUMMARY

Described herein is a framework for personalized recommendation. An image content profile for a current case is generated. One or more auxiliary information representations associated with the current case are further generated. Affinity scores for radiology service providers are then determined by applying service profiles of the radiology service providers, the image content profile and the one or more auxiliary information representations to a trained recommendation engine. The current case is then assigned to one of the radiology service providers based on the affinity scores.

### BRIEF DESCRIPTION OF THE DRAWINGS

A more complete appreciation of the present disclosure and many of the attendant aspects thereof will be readily obtained as the same becomes better understood by reference to the following detailed description when considered in connection with the accompanying drawings.

FIG. 1 shows a block diagram illustrating an exemplary system;

FIG. 2 shows an exemplary recommendation system;

FIG. 3 shows an exemplary recommendation method;

FIG. 4 shows an exemplary encoding of radiologist profiles into binary vectors;

FIG. 5 shows an exemplary encoding of radiology practice profiles into binary vectors;

FIG. 6 shows an exemplary method of generating a compact feature vector representation of an image content profile;

FIG. 7 shows an exemplary one-hot encoding vector to characterize findings in a medical image detected by a post-processing algorithm;

FIG. 8 shows an exemplary encoding of clinical information of the current patient into a compact feature vector;

FIG. 9 shows an exemplary recommendation engine;

FIG. 10 shows an exemplary personalized worklist generation; and

FIG. 11 shows another exemplary personalized worklist generation.

### DETAILED DESCRIPTION

In the following description, numerous specific details are set forth such as examples of specific components, devices, methods, etc., in order to provide a thorough understanding of implementations of the present framework. It will be apparent, however, to one skilled in the art that these specific details need not be employed to practice implementations of the present framework. In other instances, well-known materials or methods have not been described in detail in order to avoid unnecessarily obscuring implementations of the present framework. While the present framework is susceptible to various modifications and alternative forms, specific embodiments thereof are shown by way of example in the drawings and will herein be described in detail. It should be understood, however, that there is no intent to limit the invention to the particular forms disclosed, but on the contrary, the intention is to cover all modifications, equivalents, and alternatives falling within the spirit and scope of the invention. Furthermore, for ease of understanding, certain method steps are delineated as separate steps; however, these separately delineated steps should not be construed as necessarily order dependent in their performance.

Unless stated otherwise as apparent from the following discussion, it will be appreciated that terms such as “segmenting,” “generating,” “registering,” “determining,” “aligning,” “positioning,” “processing,” “computing,” “selecting,” “estimating,” “detecting,” “tracking” or the like may refer to the actions and processes of a computer system, or similar electronic computing device, that manipulates and transforms data represented as physical (e.g., electronic) quantities within the computer system's registers and memories into other data similarly represented as physical quantities within the computer system memories or registers or other such information storage, transmission or display devices. Embodiments of the methods described herein may be implemented using computer software. If written in a programming language conforming to a recognized standard, sequences of instructions designed to implement the methods can be compiled for execution on a variety of hardware platforms and for interface to a variety of operating systems. In addition, implementations of the present framework are not described with reference to any particular programming language. It will be appreciated that a variety of programming languages may be used.

One aspect of the present framework utilizes artificial intelligence (AI)-based findings as a basis for assignment of cases to create personalized worklists (or reading lists) for radiologists. If the AI engine detects a certain abnormality (e.g., suspected lung cancer) in the medical image, the present recommendation system may assign the case to be read by, for instance, a thoracic radiologist over a general radiologist. In the case of a normal scan, the present recommendation system may potentially assign the case to, for example, a general radiologist with lower relative value unit for improved cost optimization. The present recommendation system may also optimize the case load based on factors, such as availability of sub-specialty readers, criticality of the case being read, as well as environment variables (e.g., availability of radiologists, clinical reporting guidelines, current delay in reporting etc.). The present framework may be seamlessly extended to a scenario of radiology service orchestration, where cases from a radiology practice are automatically recommended to be read by an appropriate radiology service provider selected from amongst available service providers.

The present AI-based recommendation system enables dynamic generation of a radiologist personalized worklist by integration with workflow orchestration modules. The recommendation engine is built considering a multitude of information, including, but not limited to, radiology service provider profile, image information and/or clinical meta data (e.g., Electronic Health Records, clinical texts, results of automated AI-based post-processing models etc.). Upon clinical integration of such recommendation engine, this adjunctive information may be incorporated into workflow orchestration logic to ensure optimal assignment of cases to review to the appropriate radiologist at the right time.

Current workflow orchestration methods for smart case assignment to radiologists consider factors such as credentials, subspecialty, workload, availability for case-list assignment based on heuristic rules. One aspect of the present recommendation system considers personal profiles of radiologists, in addition to auxiliary inputs from post-processing algorithms, to improve case assignment, reading quality and efficiency. The present recommendation system may also be seamlessly extended for radiology service provider assignment, case assignment for peer-review and rule-out recommendation. This also allows for opportunities to set personal preferences to guide continuous education and research. These and other exemplary advantages and features will be described in more details in the following description.

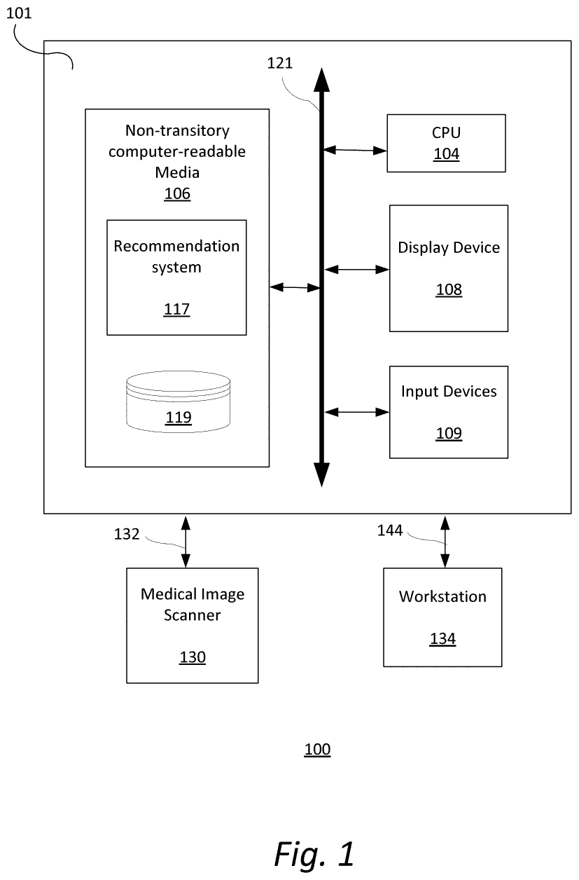

FIG. 1 is a block diagram illustrating an exemplary system 100. The system 100 includes a computer system 101 for implementing the framework as described herein. In some implementations, computer system 101 operates as a standalone device. In other implementations, computer system 101 may be connected (e.g., using a network) to other machines, such as medical image scanner 130 and workstation 134. In a networked deployment, computer system 101 may operate in the capacity of a server (e.g., in a server-client user network environment, a client user machine in server-client user network environment, or as a peer machine in a peer-to-peer (or distributed) network environment).

In one implementation, computer system 101 includes a processor device or central processing unit (CPU) 104 coupled to one or more non-transitory computer-readable media 106 (e.g., computer storage or memory device), display device 108 (e.g., monitor) and various input devices 109 (e.g., mouse, touchpad or keyboard) via an input-output interface 121. Computer system 101 may further include support circuits such as a cache, a power supply, clock circuits and a communications bus. Various other peripheral devices, such as additional data storage devices and printing devices, may also be connected to the computer system 101.

The present technology may be implemented in various forms of hardware, software, firmware, special purpose processors, or a combination thereof, either as part of the microinstruction code or as part of an application program or software product, or a combination thereof, which is executed via the operating system. In some implementations, the techniques described herein are implemented as computer-readable program code tangibly embodied in one or more non-transitory computer-readable media 106. In particular, the present techniques may be implemented by a recommendation system 117. Non-transitory computer-readable media 106 may include random access memory (RAM), read-only memory (ROM), magnetic floppy disk, flash memory, and other types of memories, or a combination thereof. The computer-readable program code is executed by CPU 104 to process data provided by, for example, database 119 and/or medical image scanner 130. As such, the computer system 101 is a general-purpose computer system that becomes a specific-purpose computer system when executing the computer-readable program code. The computer-readable program code is not intended to be limited to any particular programming language and implementation thereof. It will be appreciated that a variety of programming languages and coding thereof may be used to implement the teachings of the disclosure contained herein. The same or different computer-readable media 106 may be used for storing a database 119, including, but not limited to, image datasets, a knowledge base, individual subject data, medical records, diagnostic reports (or documents) for subjects, or a combination thereof.

Medical image scanner 130 acquires medical image data 132. Such medical image data 132 may be processed and stored in database 119. Medical image scanner 130 may be a radiology scanner (e.g., nuclear medicine scanner) and/or appropriate peripherals (e.g., keyboard and display device) for acquiring, collecting and/or storing such medical image data 132. Medical image scanner 130 may acquire the medical image data 132 by at least one of a magnetic resonance (MR) imaging, computed tomographic (CT), helical CT, x-ray, positron emission tomographic, fluoroscopic, ultrasound and single photon emission computed tomographic (SPECT) technique. Other types of modalities may also be used to acquire the medical image data 132.

The workstation 134 may include a computer and appropriate peripherals, such as a keyboard and display device, and can be operated in conjunction with the entire system 100. For example, the workstation 134 may communicate with medical image scanner 130 so that the medical image data 132 from medical image scanner 130 can be presented or displayed at the workstation 134. The workstation 134 may communicate directly with the computer system 101 to display processed data and/or output results 144. The workstation 134 may include a graphical user interface to receive user input via an input device (e.g., keyboard, mouse, touch screen, voice or video recognition interface, etc.) to manipulate visualization and/or processing of the data.

It is to be further understood that, because some of the constituent system components and method steps depicted in the accompanying figures can be implemented in software, the actual connections between the systems components (or the process steps) may differ depending upon the manner in which the present framework is programmed. Given the teachings provided herein, one of ordinary skill in the related art will be able to contemplate these and similar implementations or configurations of the present framework.

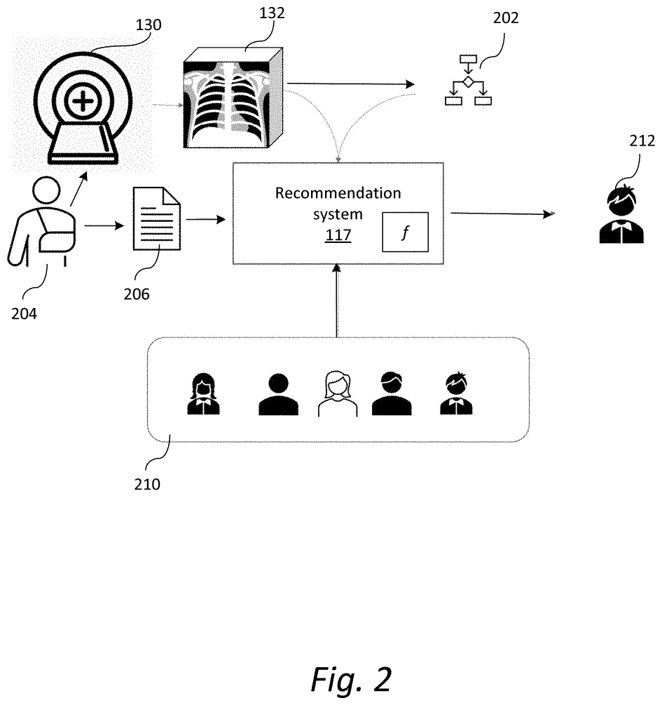

FIG. 2 shows an exemplary recommendation system 117. Recommendation system 117 uses an artificial intelligence (AI) engine 202 for automatic personalization of radiology worklists so that the case is assigned to the most appropriate radiology service provider (radiologist and/or radiology practice) 212 for reading at the right time. Recommendation system 117 facilitates automated assignment of cases to appropriate radiology service providers via a workflow orchestration process that considers latent adjunctive information. In some implementations, recommendation system 117 is designed using deep learning in such a way that each of the aforementioned factors are modeled as latent variables and the system learns the relationship that a specific case (or medical image data) 132 is best read by a particular radiology service provider 212.

Recommendation system 117 may take in, for example, clinical meta-data (or patient information) 206 associated with the patient 204, medical image data 132 of patient 204 acquired by medical image scanner 130, radiology service provider profiles 210, findings in the medical image data 132 detected by AI engine 202, radiologist preferences or environment variables as input information into a recommendation engine (f) that in turn helps assign the case for reading to the appropriate radiology service provider 212. This may be achieved after evaluating each applicable radiology service provider who is available for reading and optimizing a particular objective function based on, for example, read quality, load balancing, cost optimization, etc.

Recommendation engine f may be a deep learning model that is trained to generate an affinity score for each radiology service provider. Recommendation system 117 may then automatically populate the cases into the reading worklists of the individual radiology service providers based on, for example, their affinity scores and/or other auxiliary factors (e.g., load balance, credentials, work schedules). The cases on each worklist may be sorted according to priorities to ensure highest quality outcome. Recommendation system 117 may additionally assign cases to appropriate radiology service providers with patients they have seen or interacted with during previous visits, thereby improving patient relationship management.

Optionally, results of recommendation system 117 are sent to further downstream medical information technology (IT) systems, such as a workflow orchestration engine, Picture Archival and Communication Systems (PACS), advanced visualization or post-processing systems, radiology information system (RIS), Lab Information System (LIS), Hospital Information System (HIS), Electronic Health Record (EHR) or Electronic Medical Record (EMR), using standardized communication formats (e.g., Health Level Seven (HL7), FHIRcast).

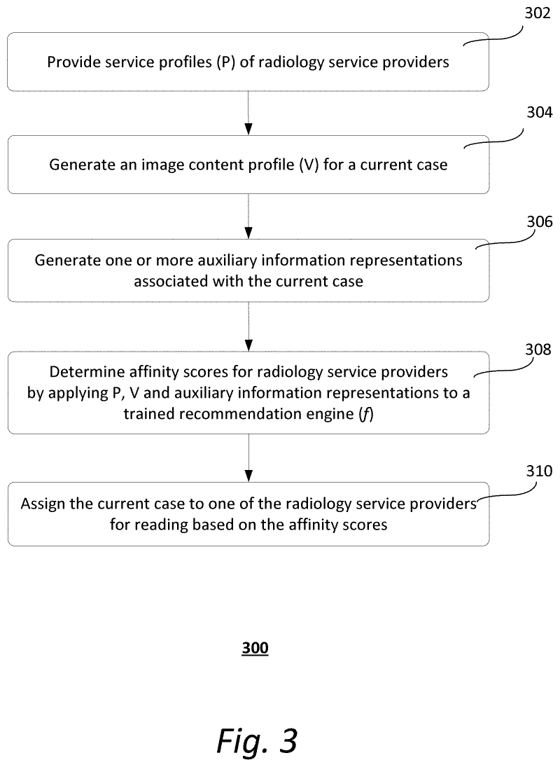

FIG. 3 shows an exemplary recommendation method 300. It should be understood that the steps of the method 300 may be performed in the order shown or a different order. Additional, different, or fewer steps may also be provided. Further, the method 300 may be implemented with the system 100 of FIG. 1, recommendation system 117 of FIG. 2, a different system, or a combination thereof.

At 302, service profiles of radiology service providers are provided using recommendation system 117. The service profile may be generated or modified each time a radiology service provider is added to or updated in the recommendation system 117. Such radiology service providers may include individual radiologists or radiology practices. In some implementations, the service profiles include radiologist profiles (U) and/or radiology practice profiles (H) that are represented by vectors (e.g., binary vectors).

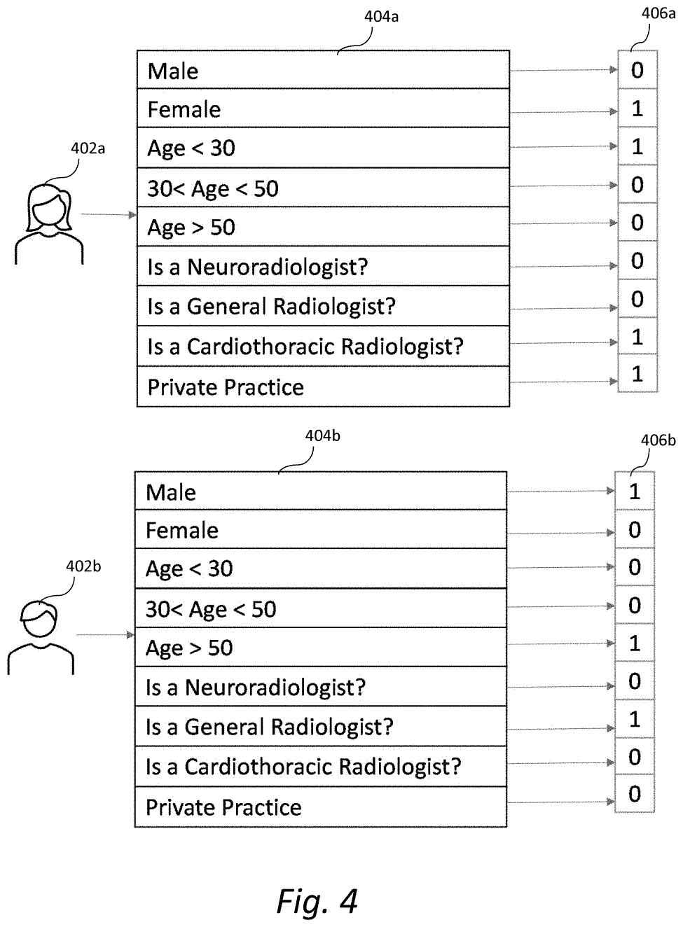

FIG. 4 shows an exemplary one-hot encoding of radiologist profiles into binary vectors (406a, 406b). Each radiologist (402a, 402b) may be characterized by a range of profile information categories (404a, 404b) including, but not limited to, gender, age, subspecialty training, experience, working in hospital settings versus private practice, based in urban center versus rural location, affinity to technology, preference of certain vendor software, etc. The profile information (404a, 404b) may be mapped to a one-hot encoding vector (406a, 406b) with integer values (e.g., 0, 1) for the categories in the profile. For example, ‘0’ may represent false and ‘1’ may represent true for the corresponding profile category (e.g., male, female, age <30) in the vector (406a, 406b). Other ways of representing radiologist profiles, such as ordinal features or continuous valued features, may also be used.

In some implementations, for serial examinations with prior reports, dynamic features representing the profile of the prior radiology service provider may be captured to ensure consistency in reporting and reduce inter-reader variability. Optionally, latent variable capturing practice constraints (e.g., balancing hard cases, workload, availability of radiologist, time of the day, seasonal disease patterns, such as flu cycles, quality control variables, etc.) may also be incorporated into the learning as features. Optionally, estimations of the radiologists' sensitivities and specificities for certain types of findings may also be incorporated into the learning. This may potentially be achieved by comparing their evaluation of AI findings to that of the majority of similar radiology service providers presented with similar AI findings.

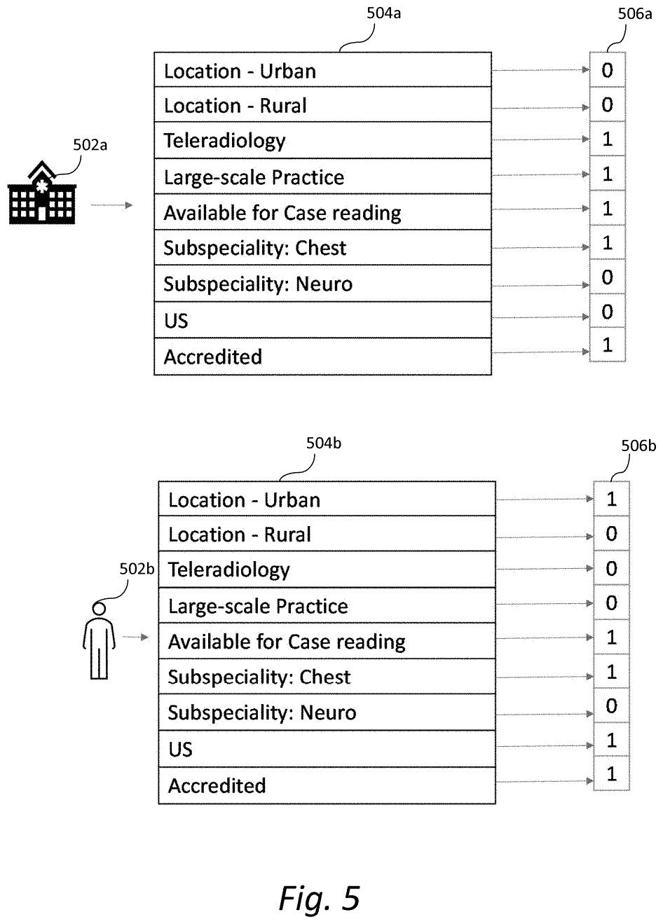

FIG. 5 shows an exemplary encoding of radiology practice profiles into binary vectors (506a, 506b). Radiology practices (502a, 502b) may include clinical institutions, solo- or multi-radiology practices. Radiology practices (502a, 502b) may be characterized by a range of radiology practice profile information (504a, 504b), including, but not limited to, nature of scale of operations, available subspecialities, current case load, geographical location (e.g., country, metropolitan, city center, suburban), accreditation status, solo- or multi-radiology practices, practice type (e.g., private, academic, teleradiology). The radiology practice profile information (504a, 504b) may be mapped to a one-hot encoding vector (506a, 506b) with integer values (e.g., 0, 1) for the categories in the radiology practice profile information. For example, ‘0’ may represent false and ‘1’ may represent true for the corresponding category (e.g., urban location, rural location, teleradiology) in the one-hot encoding vector (506a, 506b). Other ways of representing radiology practice profiles, such as ordinal features or continuous valued features, may also be used.

In the event that the hospital or imaging center where the image is acquired does not have a radiology practice or if the radiology backlog is extensive, cases may be routed to the most appropriate radiology service provider. Cases that are easier to read may be routed to teleradiology services or general radiology practices, while more complicated cases that require specialist review may be routed to specialist practices. Such a case orchestration facilitates load balancing and fewer Service Level Agreement violations. Optionally, if a case is to be referred to another radiology practice, the framework may refer it to a practice with similar prevalence of diseases, such that regional variations in disease patterns are considered while reporting.

Returning to FIG. 3, at 304, recommendation system 117 generates an image content profile (V) for a current case. The current case includes an acquired current medical image of a current patient seeking treatment. The image content profile provides information about the contents of the acquired current medical image, including image voxel values and/or meta-data. Due to the high dimensionality of the medical image, a compact feature representation of the image content profile (V) may be needed. There are a number of methods proposed for such feature extraction, including extraction of hand-crafted features (e.g., Bag of Words, Dictionary Learning, Scale Invariant Feature Transform (SIFT)) or using unsupervised deep learning based approaches such as a convolutional auto-encoder, deep clustering, deep embedding, Bayesian autoencoders, Generative Adversarial Networks, image transformers etc. or using transfer learning from pre-trained networks.

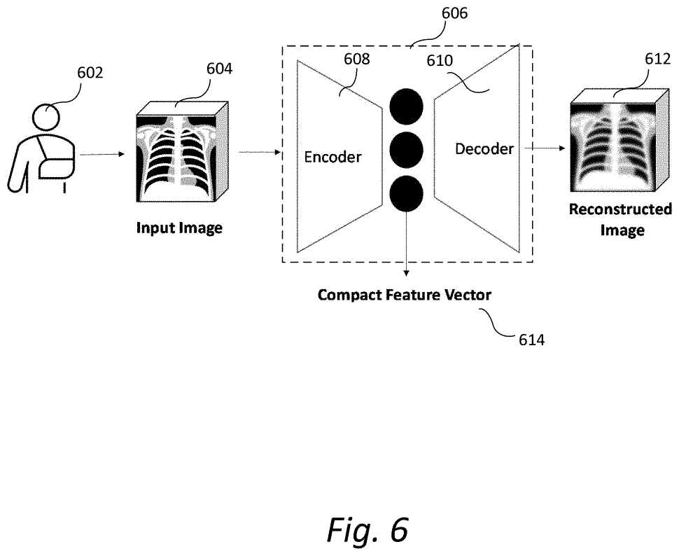

FIG. 6 shows an exemplary method of generating a compact feature vector 614 representing an image content profile 604 for current patient 602. In this illustrative example, an autoencoder-like architecture 606 takes input image 604 from one or more data sources and generates a compact feature representation 614 that can be used as a compact embedding feature vector representing the image content profile. In some implementations, one or more data sources are missing. For instance, in a multi-series magnetic resonance (MR) image, one or more optional series may be missing from the Digital Imaging and Communications in Medicine (DICOM) study. Dedicated imputation models or algorithms capable of handling missing data inputs may optionally be used in such cases. Meta-data contained in the header information, such as modality, body part examined, age, etc., may be included as features to represent the image content profile of the incoming medical image 604.

The autoencoder-like architecture 606 may include an encoder 608 and a decoder 610. Various types of autoencoder architectures may be used. Autoencoder-like architecture 606 may include, for instance, sparse autoencoders, contrastive autoencoders or autoencoders trained with adversarial loss. Other methods may also be used for encoding image content profiles 604. The encoder 608 is trained to generate a compact feature vector 614 that is used for feature representation. The decoder 610 generates a reconstructed image 612. During training of the autoencoder-like architecture 606, the reconstructed image 612 is compared to the original input image 604 to calculate a reconstruction-based loss (e.g., L2-norm). The weights in the autoencoder-like architecture 606 are iteratively updated during training to minimize the reconstruction-based loss.

Returning to FIG. 3, at 306, recommendation system 117 generates representations of one or more types of auxiliary information associated with the current case. Such auxiliary information is used to personalize the worklists generated by the recommendation system 117. Auxiliary information may include, but are not limited to, post-processing findings (I) and patient clinical profile (W). Other types of auxiliary information, such as profiles of non-radiologist readers (e.g., clinicians, residents, radiographers), may also be provided. The representations of the auxiliary information may include, for example, a one-hot encoding vector that is a binary vector that is a binary vector with a length equal to the number of categories in the data set. Alternative methods of representation, such as ordinal features or continuous valued features, may also be used.

In some implementations, post-processing findings (I) are created based on the current medical image of the current patient. The medical image may be post-processed with one or more artificial intelligence (AI) modules that analyze and detect pathological findings. Such AI findings may characterize whether the medical image has emergent or incidental findings, whether there are particular abnormalities related to major subspecialties (e.g., cardiothoracic, neurologic, abdominal, etc.). For example, if actionable findings are detected by the AI module (e.g., lung nodule detector), the case may potentially be ranked higher by the recommendation system 117 over another case involving a medical image with no actionable findings. The AI module(s) may determine, for example, preference for a specialist review in response to predicting a malignancy in the findings. The malignancy may be predicted using, for example, standardized scoring metrics, past findings or case complexity.

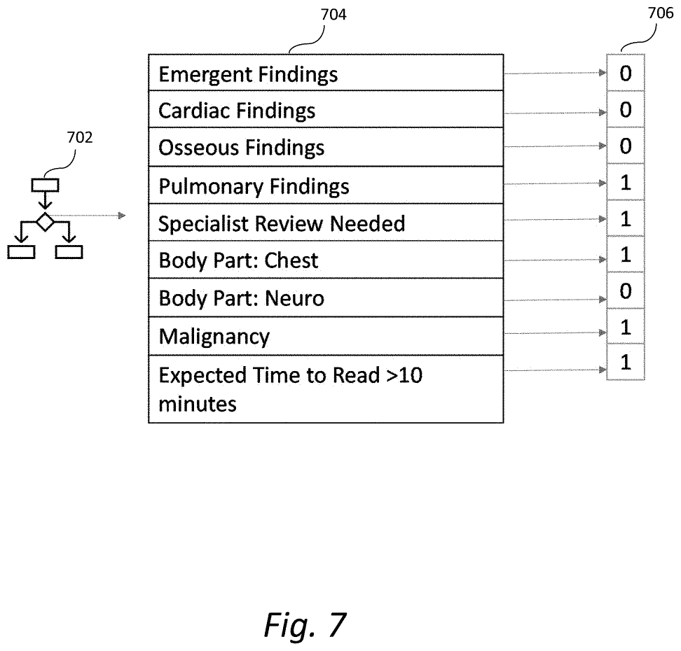

FIG. 7 shows an exemplary one-hot encoding vector 706 to characterize findings in a medical image detected by a post-processing algorithm 702. The findings may be characterized by a range of findings information categories 704, including but not limited to, whether they are emergent findings, type of findings (e.g., cardiac, osseous, pulmonary), whether specialist review is needed, body part (e.g., chest, neurology), malignancy, expected time to read (e.g., more than 10 minutes). The findings information categories 704 may be mapped to a one-hot encoding vector 706 with integer values (e.g., 0, 1).

In some implementations, a patient clinical profile (W) is created for the current patient. In a radiology reading scenario, the available patient information generally includes, but is not limited to, clinical information, image information on the current image, past scans, past clinical reports, electronic health records, etc. A patient clinical profile may be constructed considering some or all the aforementioned factors. The patient clinical profile may be represented by a compact feature vector.

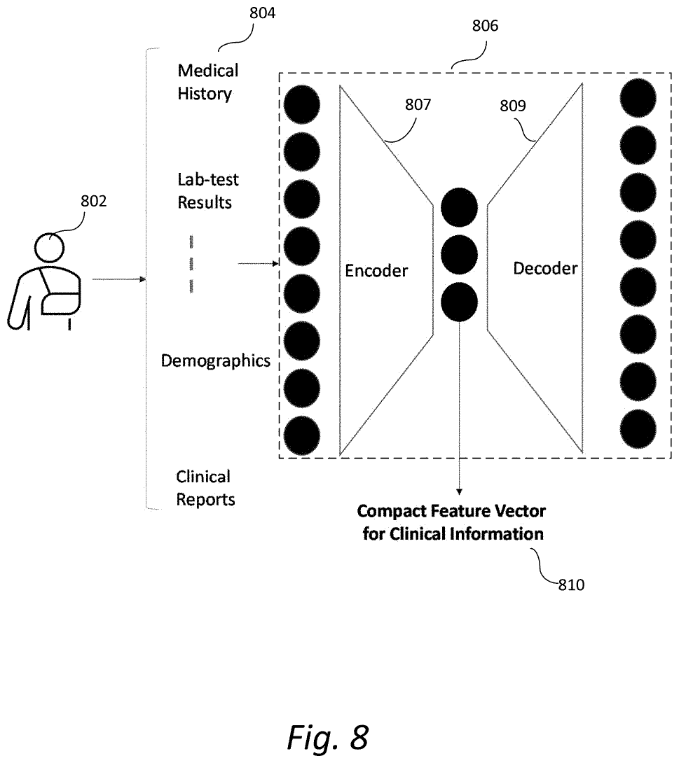

FIG. 8 shows an exemplary encoding of clinical information 804 of the current patient 802 into a compact feature vector 810. The clinical information 804 may be extracted from, for example, an electronic health record and a current scan image information. Clinical information 804 includes, for instance, medical history, laboratory test results, demographic information, clinical reports, etc. Such clinical information 804 may be encoded into a compact feature vector 810 using, for example, a deep autoencoder-like architecture 806. The deep autoencoder-like architecture 806 may include an encoder 807 and a decoder 809. Various types of deep autoencoder-like architectures may be used. Deep autoencoder-like architecture 806 may include, for instance, sparse autoencoders, contrastive autoencoders or autoencoders trained with adversarial loss. Other methods may also be used for encoding patient clinical information 804. See, for example, Shickel, B., Tighe, P. J., Bihorac, A. and Rashidi, P., Deep EHR: a survey of recent advances in deep learning techniques for electronic health record (EHR) analysis, IEEE journal of biomedical and health informatics, 22(5), pp. 1589-1604, 2017, which is herein incorporated by reference. The compact feature vector 810 may be used as additional auxiliary information for the recommendation system 117.

Returning to FIG. 3, at 310, affinity scores are determined for the radiology service providers (e.g., radiologists, radiology practices) by applying P, V and auxiliary information representations (e.g., W, I) to a trained recommendation engine (f) in the recommendation system 117. The recommendation engine (f) is trained to estimate the affinity score of each radiology service provider towards reading the current case. For radiology service provider u and incoming current case v, the affinity scoring function may be represented as: rᵤᵥ∈[0, 1], with higher value indicating higher affinity towards reading the current case. To personalize the recommendation system 117, auxiliary information x is provided as input to predict the affinity score conditioned on x:r_{uv|x} which implies the likelihood that a radiology service provider u is the best choice for reading a case v. This scoring relationship may be learnt from prior implicit or explicit user feedback to predict \hat{r}_{uv|x} by learning a recommendation engine f defined as: \hat{r}_{uv|x}=f(u, v, x, θ) where θ are learnt parameters of the recommendation system.

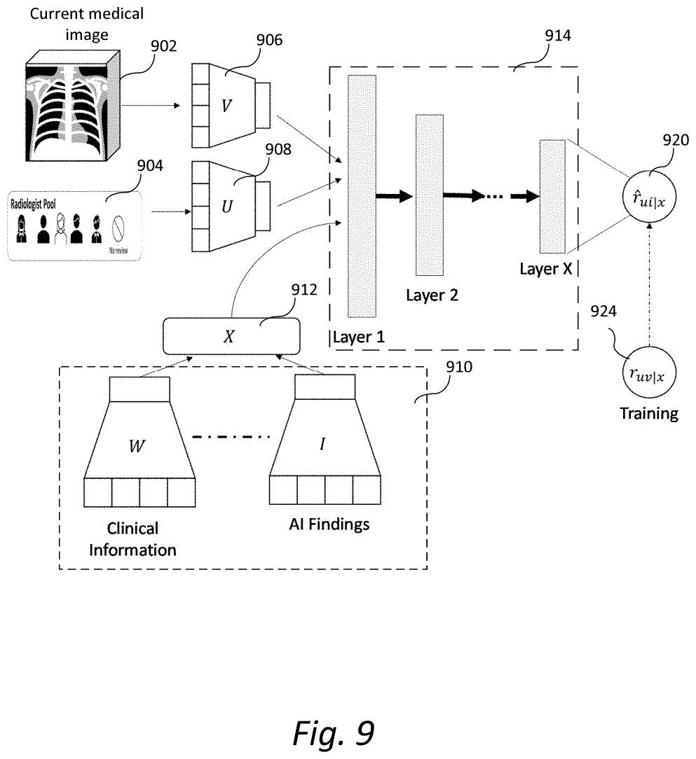

FIG. 9 shows an exemplary recommendation engine 914. Recommendation engine 914 is trained to determine the affinity scores 920 for choosing a radiology service provider u from a radiology service provider pool (904) for current medical image v (902) conditioned on auxiliary information x (912). The input to the recommendation engine 914 may include the incoming image content profile (V) 906 of a current medical image 902, radiology service provider profiles (U) 908 and a set x (912) of auxiliary information representations (910) (e.g., patient clinical profile (W), post processing findings (I)). The output of the recommendation engine 914 is a set of affinity scores 920 of the current case.

In some implementations, the recommendation engine 914 is a trained multi-view collaborative-filtering neural network, which considers, for instance, the patient's clinical information, image content information, profile of the radiology service provider and characteristics of the AI model to learn f (920). The recommendation engine 914 may be trained with weighted square loss (for explicit feedback) or with binary cross-entropy loss (for implicit feedback). Alternatively, the recommendation engine 914 may be built using methods such as deep factorization machine, wide and deep learning, deep structured semantic models, autoencoder based recommender systems etc. as illustrated by Zhang, S., Yao, L., Sun, A. and Tay, Y., Deep learning-based recommender system: A survey and new perspectives, ACM Computing Surveys (CSUR), 52(1), pp. 1-38, 2019, which is herein incorporated by reference. Optionally, to avoid cold starts that are common to recommendation systems, the recommendation engine 914 may learn directly from prior case assignment history in the institution which allows for fine-tuning to the current standard operating guidelines of the deployed institution.

In some implementations, recommendation engine 914 is operated in a closed feedback loop of affinity scores 924 for continuous learning. The continuous learning of such recommendation engine 914 may be either explicit or implicit. Explicit feedback may be provided by the user (e.g., radiologist) and include a satisfaction rating of the worklist selections provided by, for example, a ‘like’ button in a user interface generated by the recommendation system 117. Such ratings may then be used to generate feedback affinity scores 924. Indirect feedback of affinity scores 924 may be derived based on performance and quality metrics, such as reduced reading time, reduced report turn-around time, or improved quality of reporting in anonymous peer review. Such feedback may be used to improve the recommendation engine 914 using, for example, methods for weak supervision, or reinforcement learning.

Returning to FIG. 3, at 308, recommendation system 117 assigns the current case to one of the available radiology service providers for reading based on the affinity scores. For example, the radiology service provider with the highest affinity score may be assigned the current case. The worklist of the selected radiology service provider may then be populated or updated with the assigned case. The assigned radiology service provider and/or the worklist may be displayed as recommendations via a user interface (e.g., graphical user interface). The user interface may be accessed by the user via, for example, workstation 134.

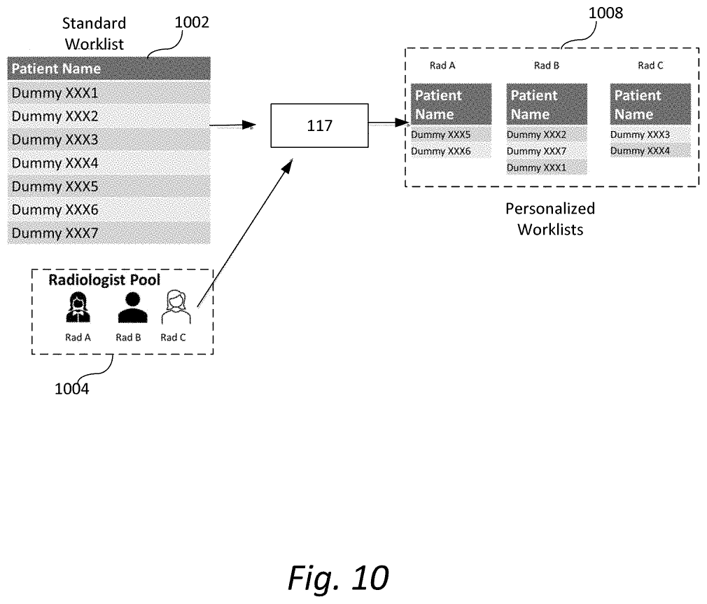

FIG. 10 shows an exemplary personalized worklist generation. In some implementations, the trained recommendation engine (f) of the recommendation system 117 is deployed as an online microservice that sweeps across all available radiologist choices in a candidate radiologist pool 1004, determines affinity score for a particular radiologist and automatically assigns the incoming current case to the top-ranked radiologist of choice and generates or automatically populates personalized radiology worklists 1008 for the radiologists with cases from the standard worklist 1002.

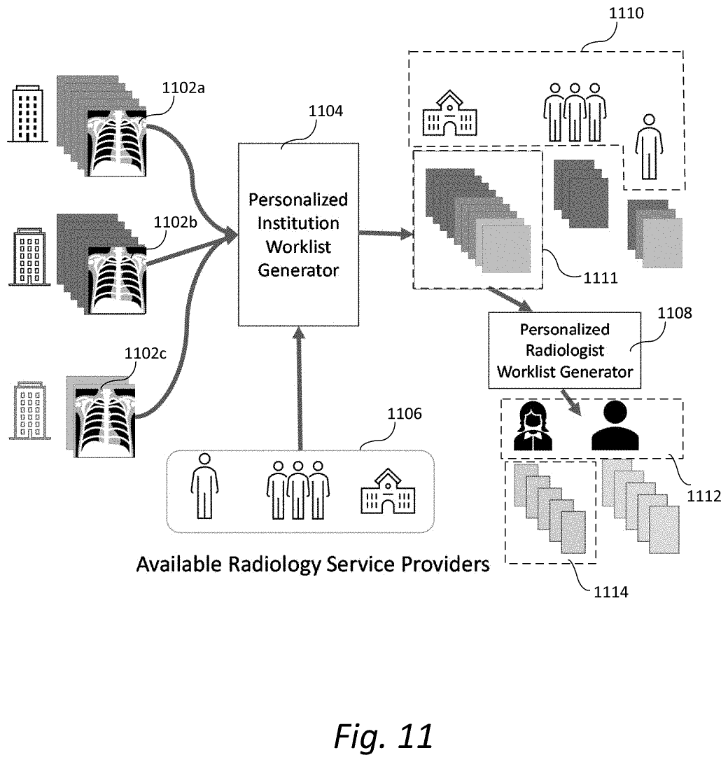

FIG. 11 shows another exemplary personalized worklist generation. Recommendation system 117 may include both a personalized institution worklist generator 1104 and a personalized radiologist worklist generator 1108. The personalized institution worklist generator 1104 assigns multiple incoming current cases (1102a, 1102b, 1102c) from different institutions or locations to the appropriate radiology practices 1110 by conditioning case allocation on radiology practice profiles 1106 of available radiology service providers. More particularly, personalized institution worklist generator 1104 may be a trained recommendation engine that determines first affinity scores for radiology practices based on service profiles 1106 of available radiology practices, image content profiles and one or more auxiliary information representations associated with one or more incoming current cases (1102a, 1102b, 1102c). Personalized institution worklist generator 1104 then assigns each of the one or more incoming current cases (1102a, 1102b, 1102c) to one of the radiology practices 1110 based on the first affinity scores. A personalized institution worklist containing the cases 1111 assigned to each of the radiology practices 1110 may then be generated.

The personalized radiologist worklist generator 1108 then further assigns the cases 1111 assigned to each radiology practice 1110 to the appropriate radiologist 1112 by conditioning case assignment on radiologist profiles of available radiologists in the radiology practice 1110. More particularly, personalized radiologist worklist generator 1108 is a trained recommendation engine that determines second affinity scores for radiologists of the radiology practice 1110 based on service profiles of the radiologists of the assigned radiology practice 1110, the image content profile and the one or more auxiliary information representations associated with the previously assigned cases 1111. Personalized radiologist worklist generator 1108 then assigns each of the cases 1111 to one of the radiologists 1112 based on the second affinity scores. A personalized radiologist worklist containing the cases 1114 assigned to each of the radiologists 1112 may then be generated.

Recommendation system 117 may optionally provide an output description, such as a textual explanation to describe one or more reasons why a particular case was assigned to a specific radiology service provider. Such description improves explainability and auditability of the system. For example, the description may explain the observance of any pattern of detecting a particular disease (e.g., interstitial lung disease), indicate that expert assessment required to rule in or rule out a particular disease pattern (e.g., usual interstitial pneumonia (UIP)), indicate that the case is assigned to a junior radiologist with patterns of lung cancer detected by AI engine, or recommend expert adjudication.

In some implementations, the trained recommendation system 117 performs evaluation for ruling out the case (i.e., Rule-Out) for any review by a radiology service provider. If the scoring function r_(u,no-reivew|x) for no review by a radiologist is rated the highest, a recommendation for Rule-Out of the case may be generated. If the scoring function r_(u,no-reivew|x) is rated higher than the scoring function r_(uv|x) across all other radiologists, the case may be recommended for Rule-Out without any radiologist review.

In some implementations, in scenarios where cases are read by more than one medical practitioner, the recommendation system 117 is modified to include, as auxiliary input, the profile of the first reader and the reported findings, so as to consider the case assignment to the appropriate second reader. This is applicable to diagnostic imaging scenarios, such as mammography, chest CT, abdominal CT, CT colonography, as well as reporting by non-radiologists (e.g., clinicians, residents, radiographers). For example, if a case was read by a resident physician, the recommendation system 117 may smartly assign the case to a specialist to ensure quality assurance and a positive teaching outcome.

While the present framework has been described in detail with reference to exemplary embodiments, those skilled in the art will appreciate that various modifications and substitutions can be made thereto without departing from the spirit and scope of the invention as set forth in the appended claims. For example, elements and/or features of different exemplary embodiments may be combined with each other and/or substituted for each other within the scope of this disclosure and appended claims.

What is claimed is:

1. A recommendation system, comprising:
one or more non-transitory computer-readable media for storing computer-readable program code; and
a processor device in communication with the one or more non-transitory computer-readable media, the processor device being operative with the computer-readable program code to perform steps including
generating an image content profile for a current case,
generating one or more auxiliary information representations associated with the current case,
determining affinity scores for radiology service providers by applying service profiles of the radiology service providers, the image content profile and the one or more auxiliary information representations to a trained recommendation engine, and
assigning the current case to one of the radiology service providers based on the affinity scores.

2. The recommendation system of claim 1 wherein the service profiles comprise radiologist profiles, radiology practice profiles, or a combination thereof.

3. The recommendation system of claim 1 wherein the image content profile comprises a compact feature representation.

4. The recommendation system of claim 1 wherein the one or more auxiliary information representations comprise one or more post-processing findings.

5. The recommendation system of claim 1 wherein the one or more auxiliary information representations comprise a patient clinical profile.

6. The recommendation system of claim 1 wherein the one or more auxiliary information representations comprise a one-hot encoding vector.

7. A recommendation method, comprising:
generating an image content profile for a current case;
generating one or more auxiliary information representations associated with the current case;
determining affinity scores for radiology service providers by applying service profiles of the radiology service providers, the image content profile and the one or more auxiliary information representations to a trained recommendation engine; and
assigning the current case to one of the radiology service providers based on the affinity scores.

8. The method of claim 7 further comprising generating the service profiles of the radiology service providers by encoding the service profiles into vectors.

9. The method of claim 8 wherein generating the service profiles comprises generating radiologist profiles, radiology practice profiles, or a combination thereof.

10. The method of claim 7 wherein generating the image content profile comprises generating a compact feature representation using an autoencoder-like architecture.

11. The method of claim 7 wherein generating the one or more auxiliary information representations associated with the current case comprises generating post-processing findings for a medical image in the current case.

12. The method of claim 11 further comprises determining, using one or more artificial intelligence modules, one or more pathological findings in the medical image.

13. The method of claim 12 further comprising determining, in response to predicting a malignancy in the one or more pathological findings, preference for a specialist review.

14. The method of claim 7 wherein generating the one or more auxiliary information representations comprises generating one or more one-hot encoding vectors.

15. The method of claim 7 wherein generating the one or more auxiliary information representations comprises generating, using an autoencoder-like architecture, one or more compact feature vectors.

16. The method of claim 7 wherein the trained recommendation engine comprises a trained collaborative-filtering neural network.

17. The method of claim 7 wherein determining the affinity scores for the radiology service providers comprises operating the trained recommendation engine in a closed feedback loop for continuous learning.

18. The method of claim 7 further comprises populating a worklist of the one of the radiology service providers with the current case.

19. The method of claim 7 further comprises generating an output description explaining one or more reasons for assigning the current case to the one of the radiology service providers.

20. One or more non-transitory computer-readable media embodying computer-readable program code executable by a processor device to perform operations comprising:
generating an image content profile for a current case;
generating one or more auxiliary information representations associated with the current case;
determining first affinity scores for radiology practices by applying service profiles of the radiology practices, the image content profile and the one or more auxiliary information representations to a first trained recommendation engine;
assigning the current case to one of the radiology practices based on the first affinity scores;
determining second affinity scores for radiologists of the one of the radiology practices by applying service profiles of the radiologists of the one of the radiology practices, the image content profile and the one or more auxiliary information representations to a second trained recommendation engine; and
assigning the current case to one of the radiologists based on the second affinity scores.
